(19) United States
(12) Patent Application Publication
(10) Pub. No.: US 2020/0354286 A1
(43) Pub. Date: Nov. 12, 2020

(54) Catalyst and Method Related Thereto

(71) Applicant: SABIC Global Technologies B.V., Bergen op Zoom (NL)

(72) Inventors: Muhammad H. Haider, Riyadh (SA); Chandrasekar Subramani, Riyadh (SA); Yasser T. Al-Harbi, Riyadh (SA); Khalid Karim, Riyadh (SA)

(21) Appl. No.: 16/764,430

(22) PCT Filed: Jan. 31, 2019
(86) PCT No.: PCT/IB2019/050797

§ 371 Date: May 15, 2020

Related U.S. Application Data
(60) Provisional application No. 62/626,435, filed on Feb. 5, 2018.

(51) Int. Cl. C07C 1/04; B01J 23/889; B01J 23/75; B01J 37/02; B01J 37/03; B01J 37/08

(52) U.S. Cl. CPC C07C 1/0445; C07C 1/043; B01J 23/8892; B01J 23/75; B01J 2523/72; B01J 37/035; B01J 37/082; B01J 2523/845; B01J 37/0236

(57) ABSTRACT

The present disclosures and inventions relate to a catalyst composition for the selective conversion of a hydrogen/carbon monoxide mixture (syngas) to C2+ hydrocarbons.

### CROSS-REFERENCE TO RELATED APPLICATIONS

This Application claims the benefit of U.S. Provisional Application No. 62/626,435, filed Feb. 5, 2018, which is incorporated herein by reference in its entirety.

### FIELD OF THE INVENTIONS

The compositions and methods disclosed herein relate to catalyst compositions and methods related thereto for the conversion of hydrogen/carbon monoxide mixtures (syngas) to hydrocarbons.

### BACKGROUND

Syngas (mixtures of H₂ and CO) can be readily produced from either coal or methane (natural gas) by methods well known in the art and widely commercially practiced around the world. A number of well-known industrial processes use syngas for producing various hydrocarbons and oxygenated organic chemicals.

The Fischer-Tropsch catalytic process for catalytically producing hydrocarbons from syngas was initially discovered and developed in the 1920's, and was used in South Africa for many years to produce gasoline range hydrocarbons as automotive fuels. The catalysts typically comprised iron or cobalt supported on alumina or titania, and promoters, like rhenium, zirconium, manganese, and the like, were sometimes used with cobalt catalysts to improve various aspects of catalytic performance. The products were typically gasoline-range hydrocarbon liquids having six or more carbon atoms, along with heavier hydrocarbon products.

Today lower molecular weight hydrocarbons are desired and can be obtained from syngas via the Fischer-Tropsch catalytic process. Challenges exist to efficiently produce C2+ hydrocarbons at high yields without producing an excess of unwanted side products.

Accordingly, there remains a long-term market need for new and improved catalysts and methods related thereto for producing increased amounts of hydrocarbons, such as C2+ hydrocarbons, from syngas. Catalysts and methods useful for the production of hydrocarbons, such as C2+ hydrocarbons, from syngas are described herein.

### SUMMARY OF THE INVENTION

Disclosed herein is a method: a) mixing a solution comprising a catalyst support and a solvent comprising water and a non-water polar solvent with an active metal composition and a precipitating agent, thereby producing a catalyst precursor.

Also disclosed herein is a catalyst prepared by the method disclosed herein.

Also disclosed herein is a composition comprising: a) a catalyst support; b) a solvent comprising water and a non-water polar solvent; c) an active metal composition; and d) a precipitating agent.

Additional advantages will be set forth in part in the description which follows, and in part will be obvious from the description, or can be learned by practice of the aspects described below. The advantages described below will be realized and attained by means of the chemical compositions, methods, and combinations thereof particularly pointed out in the appended claims. It is to be understood that both the foregoing general description and the following detailed description are exemplary and explanatory only and are not restrictive.

### DETAILED DESCRIPTION OF THE FIGURES

These and other features of the preferred embodiments of the invention will become more apparent in the detailed description in which reference is made to the appended drawing.

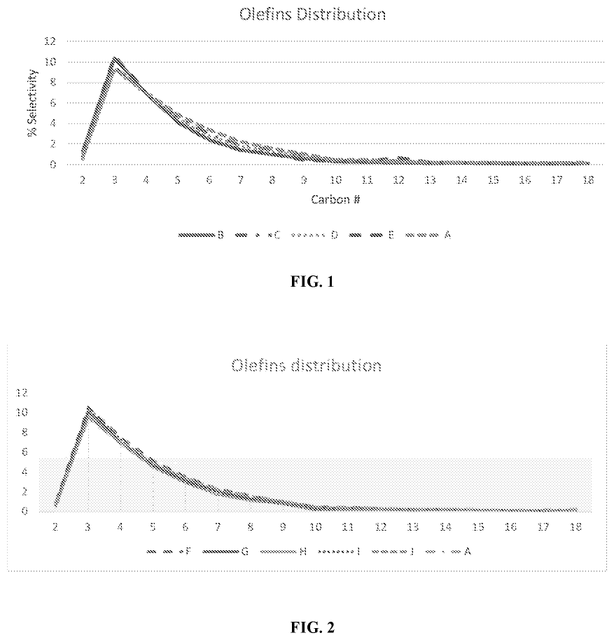

FIG. 1 shows the olefin distribution of catalysts A-E.

FIG. 2 shows the olefin distribution of catalysts A and F-J.

### DETAILED DESCRIPTION

Disclosed herein are materials, compounds, catalysts, compositions, and components that can be used for, can be used in conjunction with, can be used in preparation for, or are products of the disclosed method and compositions. It is to be understood that when combinations, subsets, interactions, groups, etc. of these materials are disclosed that while specific reference of each various individual and collective combinations and permutation of these compounds cannot be explicitly disclosed, each is specifically contemplated and described herein. For example, if a catalyst component is disclosed and discussed, and a number of alternative solid state forms of that component are discussed, each and every combination and permutation of the catalyst component and the solid state forms that are possible are specifically contemplated unless specifically indicated to the contrary. This concept applies to all aspects of this disclosure including, but not limited to, steps in methods of making and using the disclosed compositions. Thus, if there are a variety of additional steps that can be performed, it is understood that each of these additional steps can be performed with any specific aspect or combination of aspects of the disclosed methods, and that each such combination is specifically contemplated and should be considered disclosed.

In this specification and in the claims which follow, reference will be made to a number of terms which shall be defined to have the following meanings:

It must be noted that, as used in the specification and the appended claims, the singular forms “a,” “an” and “the” include plural referents unless the context clearly dictates otherwise. Thus, for example, reference to “a catalyst support” includes mixtures of catalyst supports.

“Optional” or “optionally” means that the subsequently described event or circumstance can or cannot occur, and that the description includes instances where the event or circumstance occurs and instances where it does not.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The word “or” as used herein means any one member of a particular list and also includes any combination of members of that list.

Ranges can be expressed herein as from one particular value, and/or to another particular value. When such a range is expressed, another aspect includes from the one particular value and/or to the other particular value. Similarly, when values are expressed as approximations, by use of the antecedent “,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such a ratio regardless of whether additional components are contained in the compound.

A weight percent of a component, unless specifically stated to the contrary, is based on the total weight of the formulation or composition in which the component is included.

The transitional phrase “consist essentially of” or “essentially consist of” limits the scope of the disclosure to the specified materials or steps and those that do not materially affect the basic and novel characteristic(s) of the invention.

1. Catalyst Precursor and Catalyst, and Method for Preparing Same

There is ongoing research to further develop sustainable technology of converting syngas to olefins, particularly light olefins, such as C2-C6 or C2-C4 olefins. Improving the catalyst used in this process is an important aspect of this development. Many catalytic regimes ranging from Cu, Co, La, Mn, Fe, Ni, Cr, Zr, etc. have been in focus over the past years (E. Schwab, A. Weck, J. Steiner, K. Bay, Oil Gas Eur. Mag. 1, 44-47 (2010); C. Lopez, A. Corma, Chem. Cat. Chem. 4, 751-752 (2012); M. E. Dry, “The Fischer-Tropsch process: 1950-2000” Catalysis Today, vol. 71, pp. 227-241, January 2002). Cobalt based catalysts are of particular interest as they show efficient activity at low temperatures i.e. high conversions and long-term stability as compared to other catalyst regimes (F. Diehl, and A. Y. Khodakov, “Promotion of Cobalt Fischer-Tropsch Catalysts with Noble Metals: a Review,” Oil Gas Sci. Technol.-Rev. IFP vol. 64, no. 1, pp. 11-24, November 2008; Vannice, M. A. J. Catal. 1975, 37, 449). Different attempts have been made to further enhance and improve the efficiency and selectivity towards desired products to improve the cobalt based catalyst regime (James Aluha et al Industrial & Engineering Chemistry Research 2015 54 (43), 10661-10674; Gregory R. Johnson et al ACS Catalysis 2015 5 (10), 5888-5903). Disclosed herein is a method of producing a catalyst, such as a cobalt based catalyst, that produces an improved catalyst, such as an improved cobalt catalyst. The disclosed method involves the use of a solvent medium that in turn affects the performance of cobalt catalyst in terms of activity and selectivity towards desired products, such as light olefins, such as C2-C6 or C2-C4 olefins.

Disclosed herein is a method of preparing a catalyst precursor. Also disclosed, herein is a precursor catalyst prepared by the disclosed method. In one aspect, the catalyst precursor is a catalyst precursor suitable for use in a Fischer-Tropsch reaction. In one aspect, the catalyst precursor is a CoMn catalyst precursor. It is understood that the CoMn catalyst precursor is present in or on the catalyst support and that the catalyst support is also a part of the catalyst precursor.

Also, disclosed herein is a method of preparing a catalyst. Also disclosed, herein is a catalyst prepared by the disclosed method. In one aspect, the catalyst precursor is a catalyst suitable for use in a Fischer-Tropsch reaction. In one aspect, the catalyst is a CoMn catalyst. It is understood that the CoMn catalyst is present in or on the catalyst support and that the catalyst support is also a part of the catalyst.

In one aspect, the CoMn catalyst precursor has the formula CoMnₓS_{y}O_{z} catalyst precursor, wherein S is a catalyst support. It is understood that the catalyst support is a part of the catalyst precursor. In one aspect, the CoMn catalyst has the formula CoMnₓS_{y}O_{z} catalyst, wherein S is a catalyst support. It is understood that the catalyst support is a part of the catalyst.

The disclosed catalyst is for converting syngas to hydrocarbons, for example, selectively converting syngas to C2+ hydrocarbons, such as, for example, C2-C6 hydrocarbons or C2-C4 hydrocarbons. The catalyst disclosed herein has an improved conversion rate and selectivity for converting syngas to C2+ hydrocarbons, such as, for example, C2-C6 hydrocarbons or C2-C4 hydrocarbons, as compared to conventional catalysts.

In the composition comprising the CoMnₓS_{y}O_{z} catalyst, the molar ratio of manganese atoms to cobalt atoms, i.e. the value of “x” in the catalyst formula, can be from about 0.8 to about 1.2, from about 0.8 to about 1.1, from about 0.8 to about 1.0, from about 0.8 to about 0.9, from about 0.9 to about 1.2, from about 0.9 to about 1.1, from about 0.9 to about 1.0, from about 1.0 to about 1.2, or from about 1.0 to about 1.1. In one aspect, x can be about 1.0.

In the composition comprising the CoMnₓS_{y}O_{z} catalyst, the molar ratio of the catalyst support “S” atoms to cobalt atoms, i.e. the value of “y” in the catalyst formula, can be from about 0.01 to about 5.0, from about 0.1 to about 3.0, from about 0.1 to about 1.0, from about 0.3 to about 1.0, from about 0.5 to about 1.0, from about 0.7 to about 1.0, from about 0.1 to about 0.8, from about 0.3 to about 0.8, or from about 0.1 to about 0.5. In one aspect, y can be about 1.0 or about 0.5.

In one aspect, the molar ratio of x can be about 1.0 and the molar ratio of y can be from about 0.1 to about 1.0. In another aspect, the molar ratio of x can be from about 0.9 to about 1.1 and the molar ratio of y can be from about 0.1 to about 1.0. In yet another aspect, the molar ratio of x can be from about 0.9 to about 1.1 and the molar ratio of y can be from about 0.1 to about 0.8. In yet another aspect, the molar ratio of x can be from about 0.9 to about 1.1 and the molar ratio of y can be from about 0.5 to about 1.0.

In the composition comprising the CoMnₓS_{y}O_{z} catalyst, the molar ratio of oxygen atoms, i.e. the value of “z” in the catalyst formula, is a number determined by the valence requirements of Co, Mn, and catalyst support “S.” In one aspect, z is greater than 0 (zero). In another aspect, z can be 0 (zero). Even though a suitable catalyst composition of these inventions may be prepared or loaded into a reactor in the form of a mixed oxide (i.e. z is initially greater than 0), contact with hot syngas, either before or during the catalytic conversion of syngas to hydrocarbons begins, may result in the “in-situ” reduction of the catalyst composition and/or partial or complete removal of oxygen from the solid catalyst composition, with the result that z can be decreased to zero or zero. In one aspect, the value of z can be any whole integer or decimal fraction between 0 and 10. In some aspects of the catalyst described herein, z is greater than zero. In some aspects of the catalysts described herein, z can be from 1 to 5.

Also disclosed herein is a composition comprising the disclosed catalyst precursor and a catalyst support material. Also disclosed herein is a composition comprising the disclosed catalyst and a catalyst support material.

The composition comprising a catalyst having the formula CoMnₓS_{y}O_{z} disclosed herein have a low water gas shift activity as compared to conventional catalyst. The water gas shift reaction provides a source of H₂ and CO₂ at the expense of CO and H₂O. Thus, unwanted CO₂ is produced by the water gas shift reaction. The composition comprising a catalyst having the formula CoMnₓS_{y}O_{z} disclosed herein have a low water gas shift activity, thereby producing a low amount of CO₂ as shown herein. For example, the composition comprising a catalyst having the formula CoMnₓS_{y}O_{z} disclosed herein have a water gas shift reaction that produces less than 10% or less than 6% CO₂ from the carbon monoxide feed. Accordingly, the composition comprising a catalyst having the formula CoMnₓS_{y}O_{z} disclosed herein can have a CO₂ selectivity that is less than 10% or less than 5%.

In one aspect, the composition consists essentially of a catalyst precursor or a catalyst having the formula CoMnₓS_{y}O_{z}, wherein the molar ratio of x is from about 0.8 to about 1.2; wherein the molar ratio of y is from about 0.01 to about 5.0; and wherein the molar ratio of z is a number determined by the valence requirements of Co, Mn, and the catalyst support “S”, and a catalyst support. For example, the composition can consist essentially of a catalyst precursor or a catalyst having the formula CoMnₓS_{y}O_{z}, wherein the molar ratio of x is from about 0.9 to about 1.1; wherein the molar ratio of y is from about 0.1 to about 1.0; and wherein the molar ratio of z is a number determined by the valence requirements of Co, Mn, and the catalyst support “S.”

The CoMnₓS_{y}O_{z} catalyst precursor catalyst and/or CoMnₓS_{y}O_{z} catalyst herein can be non-stoichiometric solids, i.e. single phase solid materials whose composition cannot be represented by simple ratios of well-defined simple integers, because those solids probably contain solid state point defects (such as vacancies or interstitial atoms or ions) that can cause variations in the overall stoichiometry of the composition. Such phenomena are well known to those of ordinary skill in the arts related to solid inorganic materials, especially for transition metal oxides. Accordingly, for convenience and the purposes of this disclosure, the composition of the potentially non-stoichiometric catalytically active solids described herein will be quoted in ratios of moles of the other atoms as compared to the moles of cobalt and manganese ions or atoms in the same composition, whatever the absolute concentration of cobalt and manganese present in the composition. Accordingly, for purposes of this disclosure, the value of “x” and “y” are molar ratios relative to each other, regardless of the absolute concentration of cobalt and manganese in the catalyst. Thus, the subscript numbers represents molar ratios.

In one aspect, the composition comprising the CoMnₓS_{y}O_{z} catalyst precursor or the CoMnₓS_{y}O_{z} catalyst, wherein S is a catalyst support, the catalyst support is typically catalytically inert, but typically provides physical support, strength and integrity to catalyst particles or pellets containing both the catalyst compositions and the catalyst supports, so that catalyst lifetimes and performances are improved. Suitable catalyst supports (“S”) for the CoMnₓS_{y}O_{z} catalyst precursor and CoMnₓS_{y}O_{z} catalyst comprises Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof. For example, S can comprise Al₂O₃, SiO₂, TiO₂, or ZrO₂. In another example, S can comprise SiO₂.

In one aspect, the composition essentially consists of the CoMnₓS_{y}O_{z} catalyst precursor. In another aspect, the composition consists of the CoMnₓS_{y}O_{z} catalyst.

In one aspect, the composition essentially consists of the CoMnₓS_{y}O_{z} catalyst. In another aspect, the composition consists of the CoMnₓS_{y}O_{z} catalyst.

Accordingly, disclosed herein is a method: a) mixing a solution comprising a catalyst support and a solvent comprising water and a non-water polar solvent with an active metal composition and a precipitating agent, thereby producing a catalyst precursor.

The use of a solvent with a non-water polar solvent, such as at least on alcohol, to water and mixing it to an elevated temperature, for example 80° C., leads to the formation of ordered bi-phase (binary) environment around the catalyst support allowing the active metal particles (i.e. Co and Mn) to form a specified pattern. This pattern can be nano-routed as the organic portion of the solvent will interfere with the surface of the catalyst support making the size of the surface channels specific in nature. The active metal particles filter through this in-situ sieve type medium and achieve a uniform porosity in the finished catalyst, which structure leads to increased activity of the catalysts.

In one aspect, the method further comprises the steps of: b) drying the catalyst precursor; and c) calcining the catalyst precursor, thereby producing a catalyst.

Accordingly, also disclosed herein is a composition comprising: a) a catalyst support; b) a solvent comprising water and a non-water polar solvent; c) an active metal composition; and d) a precipitating agent.

It is understood that the components in the disclosed composition can be further defined as described herein. For example, the catalyst support can comprise Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof; the non-water polar solvent can comprise at least one alcohol; the active metal composition can comprise cobalt and manganese; and the precipitating agent can contain a carbonate, bicarbonate, phosphate, or borate.

The disclosed method uses a solvent comprising water and a non-water polar solvent. In one aspect, the solvent is a binary solvent comprising water and a non-water polar solvent. In another aspect, the solvent consists essentially of water and a non-water polar solvent.

In one aspect, the solvent comprises a volume ratio of the water:non-water polar solvent in the solvent is from about 1:10 to about 10:1. For example, the solvent can comprise a volume ratio of the water:non-water polar solvent in the solvent is from about 1:7 to about 7:1. In another example, the solvent can comprise a volume ratio of the water:non-water polar solvent in the solvent is from about 1:5 to about 5:1. In yet another example, the solvent can comprise a volume ratio of the water:non-water polar solvent in the solvent is from about 10:1 to about 1:1. In yet another example, the solvent can comprise a volume ratio of the water:non-water polar solvent in the solvent is from about 7:1 to about 3:1.

In yet another example, the solvent can comprise a volume ratio of the water:non-water polar solvent in the solvent is from about 1:10 to about 1:1. In yet another example, the solvent can comprise a volume ratio of the water:non-water polar solvent in the solvent is from about 1:8 to about 1:2. In yet another example, the solvent can comprise a volume ratio of the water:non-water polar solvent in the solvent is from about 1:7 to about 1:3.

In yet another example, the solvent can comprise a volume ratio of the water:alcohol in the solvent is from about 1:10 to about 1:1. In yet another example, the solvent can comprise a volume ratio of the water:alcohol in the solvent is from about 1:8 to about 1:2. In yet another example, the solvent can comprise a volume ratio of the water:alcohol in the solvent is from about 1:7 to about 1:3.

In one aspect, the non-water polar solvent comprises at least one alcohol. For example, the non-water polar solvent can consist essentially of one alcohol. In one aspect, the at least one alcohol comprises methanol, ethanol, butanol, butane diol, pentanol, hexanol, ethylene glycol, propylene glycol, dipropylene glycol, tripropylene glycol, and butylene glycol, or a mixture thereof. For example, the at least one alcohol can comprise ethanol, butanol, butane diol, or ethylene glycol. In another example, the at least one alcohol can comprise butanol.

In another aspect the non-water polar solvent comprises an acid, such as acetic acid. In yet another aspect the non-water polar solvent comprises dimethyl sulfoxide (DMSO), an acetate, acetone, or tetrahydrofuran (THF), or a combination thereof. In another aspect the non-water polar solvent consists essentially of an acid, such as acetic acid. In yet another aspect the non-water polar solvent consists essentially of dimethyl sulfoxide (DMSO), an acetate, acetone, or tetrahydrofuran (THF), or a combination thereof.

It is understood that residual amounts of water can be present in the non-water polar solvent. For example, there is typically some amount of water present in the at least one alcohol, such as methanol, ethanol, butanol, butane diol, pentanol, hexanol, ethylene glycol, propylene glycol, dipropylene glycol, tripropylene glycol, and butylene glycol, or a mixture thereof.

In one aspect, the wherein the solution has a temperature from about 20° C. to about 95° C. For example, the solution can have a temperature from about 40° C. to about 95° C. In another example, the solution can have a temperature from about 60° C. to about 95° C. In yet another example, the solution can have a temperature from about 70° C. to about 95° C. In yet another example, the solution can have a temperature from about 70° C. to about 90° C. In yet another example, the solution can have a temperature from about 75° C. to about 85° C.

In one aspect, the concentration of the catalyst support in the solution is at least about 0.0001 g catalyst support per ml of solvent. For example, the concentration of the catalyst support in the solution can be at least about 0.001 g catalyst support per ml of solvent. In another example, the concentration of the catalyst support in the solution can be at least about 0.005 g catalyst support per ml of solvent. In yet another example, the concentration of the catalyst support in the solution can be from about 0.001 g to about 0.05 g catalyst support per ml of solvent.

In one aspect, the precipitating agent comprises a carbonate, bicarbonate, phosphate, or borate. For example, the precipitating agent can be a water soluble carbonate, bicarbonate, phosphate, or borate. In another example, the precipitating agent can comprise, for example, water soluble carbonates and bicarbonates of ammonium, sodium, potassium, lithium, and cesium. In yet another example, the precipitating agent can comprise ammonium carbonate or sodium carbonate.

In one aspect, the method comprises adjusting the pH of the solution from acidic or neutral to basic. For example, the method can comprise adjusting the pH of the solution from acidic or neutral to about 8.5. In another example, the method can comprise adjusting the pH of the solution from about 7.0 to about 8.0. In one aspect, the solution has a pH from 6.5 to about 8.5.

In one aspect, the active metal composition comprises a cobalt and manganese. The active metal composition determines to composition of the catalyst precursor and catalyst. For example, when the active metal composition comprises a cobalt and manganese a CoMnₓS_{y}O_{z} catalyst precursor and CoMnₓS_{y}O_{z} catalyst can be obtained. Many suitable compounds comprising Co that are soluble in water can be suitable. In one aspect, a solvent comprises water and at least one alcohol. Any cobalt (II) or (III) salt that is soluble in the solvent, can be used, and the use of cobalt (II) nitrate, cobalt tris(acetylacetonate), cobalt bis(acetylacetonate), cobalt (II) chloride, cobalt (II) bromide, cobalt (II) iodide, cobalt (II) acetate, cobalt (II) sulfate_ and cobalt (II) diacetate, or a combination thereof are a specific examples of a suitable Co compound that can be dissolved to provide a suitable solution comprising Co. Any manganese (II) or (III) salt that is soluble in the solvent can be used, and the use of manganese (II) nitrate or manganese (II) acetate are a specific examples of suitable Mn compounds that can be dissolved to provide a suitable solution comprising Mn.

In one aspect, the solution comprises from about 0.1 mole % to about 2.0 mole %, such as for example, from about 0.5 mole % to about 1.5 mole %, of the cobalt salt prior to the formation of the CoMnₓS_{y}O_{z} catalyst precursor. In another aspect, the solution comprises from about 0.1 mole % to about 2.0 mole %, such as for example, from about 0.5 mole % to about 1.5 mole %, of the manganese salt prior to the formation of the CoMnₓS_{y}O_{z} catalyst precursor.

In one aspect, the method further comprises drying the CoMnₓS_{y}O_{z} catalyst precursor. The drying of the CoMnₓS_{y}O_{z} catalyst precursor can be done at a temperature from about 90° C. to about 180° C., such as, for example, from about 110° C. to about 150° C. In another aspect, the CoMnₓS_{y}O_{z} catalyst precursor is not filtered or washed prior to the drying step. One advantage of using ammonium carbonate as a precipitating agent is that the CoMnₓS_{y}O_{z} catalyst precursor can be processed without first having to filter and/or wash the CoMnₓS_{y}O_{z} catalyst precursor. Of course, in one aspect, the CoMnₓS_{y}O_{z} catalyst precursor can still be filtered, washed, and dried.

In one aspect of the methods for making the catalyst compositions, the method further comprises calcining the CoMnₓS_{y}O_{z} catalyst precursor, thereby producing a CoMnₓS_{y}O_{z} catalyst. The calcining can be done in the presence of oxygen or air at high temperatures (such as for example exposing the catalyst composition to a temperature of from, about 200° C. to about 800° C.), or similar heating under a dry inert gas such as nitrogen, can also be required in order to fully form the catalyst compositions. For example, calcining can result in the conversion of a physical mixture of components to form the catalyst phase, via various chemical reactions, such as for example the introduction of oxygen atoms or ions into the composition. In one aspect, the method further comprises calcining the dried CoMnₓS_{y}O_{z} catalyst precursor at a temperature from about 350° C. to about 650° C., to produce a CoMnₓS_{y}O_{z} catalyst.

As shown and described herein, the catalyst, such as a CoMnₓS_{y}O_{z} catalyst, resulting from the method disclosed herein using a solvent comprising water and a non-water polar solvent surprisingly has improved properties, such as, improved conversion rate and selectivity for converting syngas to C₂-C₆ hydrocarbons, such as, for example, C₂-C₄ hydrocarbons, as compared to a catalyst, such as a CoMnₓS_{y}O_{z} catalyst, prepared using a conventional solvent.

It is also to be understood that in some aspects of the compositions and methods described herein, once a catalyst has been formed by the methods described above, and the formed catalyst is loaded into reactors and contacted with syngas at reaction temperatures for significant periods of time, some physical and chemical changes can occur in the catalyst, either quickly or over time as the catalytic reactions with syngas are carried out. For example, contact of the metal oxide catalysts described herein with syngas at high temperatures can cause partial or complete “in-situ” reduction of the metal oxides, and such reduction processes can cause removal of oxygen atoms from the solid catalyst lattices, and/or cause reduction of some or all of the metal cations present in the catalyst to lower oxidation states, including reduction to metallic oxidation states of zero, thereby producing finely divided and/or dispersed metals on the catalyst supports. Such reduced forms of the catalysts of the invention are within the scope of the described compositions and methods.

The possible components and ranges of components for such compositions have already been described above, and can be applied in connection with describing and claiming methods for preparing such compositions.

In view of the general descriptions of the preparations of the catalyst compositions and variations thereof that are part of these inventions described above, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

2. Methods for Producing Hydrocarbons from Syngas

Described above is a composition comprising a catalyst having the generic formula CoMnₓS_{y}O_{z} catalyst and methods for making such a catalyst. The catalyst is useful for converting mixtures of carbon monoxide and hydrogen (syngas) to hydrocarbons. The catalyst has unexpectedly high conversions of CO and selectivity for converting syngas to C2+ hydrocarbons, such as to low molecular weight hydrocarbons such as C₂-C₆ hydrocarbons, such as, C₂-C₄ hydrocarbons.

Also disclosed herein is a method of producing C2+ hydrocarbons comprising contacting syngas with a composition comprising a catalyst having the formula CoMnₓS_{y}O_{z} catalyst, as disclosed herein, thereby producing C2+ hydrocarbons, such as C₂-C₆ hydrocarbons, such as, C₂-C₄ hydrocarbons.

The catalyst composition has a formula comprising CoMnₓS_{y}O_{z} catalyst prior to introducing it to conditions suitable for contacting and reacting the catalyst composition with the syngas. Such conditions are known in the art and include high temperatures. The catalyst composition is reduced when present in the conditions associated with process of producing C2+ hydrocarbons by contacting the catalyst composition with syngas. Such catalyst composition is and can be referred to herein as a “reduced form of a catalyst composition comprising.” A reduction of the catalyst compositions under such conditions is known to those skilled in the art.

In these methods, mixtures of carbon monoxide and hydrogen (syngas) are contacted with suitable catalysts (whose composition, characteristics, and preparation have been already described above and in the Examples below) in suitable reactors and at suitable temperatures and pressures, for a contact time and/or at a suitable space velocity needed in order to convert at least some of the syngas to hydrocarbons. Unexpectedly as compared to methods in the prior art, the methods of the present inventions can be highly selective for the production of C2+ hydrocarbons, which are valuable feedstocks for subsequent cracking processes at refineries for producing downstream products, such as low molecular weight olefins. C2+ hydrocarbons can be C₂-C₁₂ hydrocarbons, C₂-C₈ hydrocarbons, C₂-C₆ hydrocarbons, C₂-C₄ hydrocarbons or C₂-C₃ hydrocarbons.

Methods for producing syngas from natural gas, coal, or waste streams or biomass, at almost any desired ratio of hydrogen to carbon monoxide are well known to those of ordinary skill in the art. A large range of ratios of hydrogen to carbon monoxide can be suitable for the practice of the current invention, but since high conversion of carbon monoxide to hydrocarbons is desired, syngas mixtures comprising at least equimolar ratios of hydrogen to carbon monoxide or higher are typically employed, i.e. from 3:1 H₂/CO to 1:1 H₂/CO. In some aspects, the ratios of hydrogen to carbon monoxide employed are from 2:1 H₂/CO to 1:1 H₂/CO. Optionally, inert or reactive carrier gases, such as N₂, CO₂, methane, ethane, propane, and the like can be contained in and/or mixed with the syngas.

The syngas is typically forced to flow through reactors comprising the solid catalysts, wherein the reactors are designed to retain the catalyst against the vapor phase flow of syngas, at temperatures sufficient to maintain most of the hydrocarbon products of the catalytic reactions in the vapor phase at the selected operating pressures. The catalyst particles can be packed into a fixed bed, or dispersed in a fluidized bed, or in other suitable arrangements known to those of ordinary skill in the art.

In one aspect, the syngas is contacted with the catalyst compositions at a temperature of at least 200° C., or at least 300° C., and at a temperature below 400° C. or from a temperature of 200° C. to 350° C., or from a temperature of 230° C. to 270° C.

In one aspect, the syngas is contacted with the catalyst compositions at a pressure of at least 3 bar, 5 bar, or at least, 10 bar, or at least 15 bar, or at least 25 bar, or at least 50 bar, or at least 75 bar, and less than 200 bar, or less than 100 bar. In many aspects of the methods of the reaction, the syngas is contacted with the catalyst compositions at a pressure from 5 bar to 100 bar. In many aspects of the methods of the reaction, the syngas is contacted with the catalyst compositions at a pressure from about 3 bar to about 15 bar.

In one aspect, the syngas is contacted with the catalyst compositions to produce relatively high conversions of the carbon monoxide present in syngas. In one aspect, conversion of carbon monoxide is at least 60%, at least 65%, at least 67%, at least 70%, at least 73%, or at least 75%. In one aspect, less than 30%, or less than 25% of the carbon monoxide fed to the reactors is converted to CO₂.

In one aspect, the methods disclosed herein are unexpectedly highly selective for the production of C2+ hydrocarbons. Typical C2+ hydrocarbons, detected in the product include saturated hydrocarbons such as methane, ethane, propanes, butanes, and pentanes, and unsaturated hydrocarbons such as ethylene, propylene, butenes, and pentenes. In another aspect, the methods disclosed herein are unexpectedly highly selective for the production of C2+ olefins, such as propylene. Typical C2+ olefins, detected in the product include ethylene, propylene, butenes, and pentenes. In one aspect, the method has an unexpectedly higher selectivity as compared to a reference catalyst not being prepared with a conventional solvent.

In one aspect, the selectivity for production of olefins can be from about 30% to about 40%, from about 32% to about 37%. In one aspect, the selectivity for production of propylene can be from at least about 10%, for example, from about 10% to about 15%.

The production of methane in a Fischer-Tropsch process is undesired. In one aspect, the selectivity for production of methane can be less than about 10%, less than about 8%, or less than about 7%.

In view of the general descriptions of the catalyst compositions and variations thereof that are part of the inventions described above, herein below are described certain more particularly described aspects of methods for employing the catalysts for converting syngas to hydrocarbons. These particularly recited aspects should not however be interpreted to have any limiting effect on any different claims containing different or more general teachings, or that the “particular” aspects are somehow limited in some way other than the inherent meanings of the language and formulas literally used therein.

3. Aspects

In view of the described catalyst and catalyst compositions and methods and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A method comprising the step of: a) mixing a solution comprising a catalyst support and a solvent comprising water and a non-water polar solvent with an active metal composition and a precipitating agent, thereby producing a catalyst precursor.

Aspect 2: The method of aspect 1, wherein the method further comprises the steps of: b) drying the catalyst precursor; and c) calcining the catalyst precursor, thereby producing a catalyst.

Aspect 3: The method of aspects 1 or 2, wherein the solution has a temperature from about 20° C. to about 95° C.

Aspect 4: The method of aspects 1 or 2, wherein the solution has a temperature from about 60° C. to about 90° C.

Aspect 5: The method of any one of aspects 1-4, wherein the catalyst support comprises Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof.

Aspect 6: The method of aspect 5, wherein the catalyst support comprises SiO₂.

Aspect 7: The method of any one of aspects 1-6, wherein the non-water polar solvent comprises at least one alcohol.

Aspect 8: The method of aspect 7, wherein the at least one alcohol comprises methanol, ethanol, butanol, butane diol, pentanol, hexanol ethylene glycol, propylene glycol, dipropylene glycol, tripropylene glycol, or butylene glycol, or a mixture thereof.

Aspect 9: The method of aspect 7, wherein the at least one alcohol comprises butanol.

Aspect 10: The method of any one of aspects 1-9, wherein the volume ratio of the water:non-water polar solvent in the solvent is from about 1:10 to about 1:1.

Aspect 11: The method of any one of aspects 1-10, wherein the concentration of the catalyst support in the solution is at least 0.0001 g catalyst support per ml of solvent.

Aspect 12: The method of any one of aspects 1-11, wherein the active metal composition comprises a cobalt and manganese.

Aspect 13: The method of any one of aspects 1-12, wherein the precipitating agent comprises a carbonate, bicarbonate, phosphate, or borate.

Aspect 14: The method of any one of aspects 1-3, wherein the solution has a basic pH.

Aspect 15: The method of any one of aspects 1-14, wherein the catalyst has the formula CoMnₓS_{y}O_{z}, wherein S is a catalyst support, wherein the molar ratio of x is from about 0.8 to about 1.2; wherein the molar ratio of y is from about 0.01 to about 5.0; and wherein the molar ratio of z is a number determined by the valence requirements of Co, Mn, and S.

Aspect 16: A catalyst precursor produced by the method of any one of aspects 1-15.

Aspect 17: A catalyst produced by the method of any one of aspects 2-15.

Aspect 18: A method of producing C2+ hydrocarbons comprising contacting syngas with the catalyst of any one of aspects 2-15, thereby producing C2+ hydrocarbons.

Aspect 19: A composition comprising: a) a catalyst support; b) a solvent comprising water and a non-water polar solvent; c) an active metal composition; and d) a precipitating agent.

Aspect 20: The composition of aspect 19, wherein a) the catalyst support comprises Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof; b) the non-water polar solvent comprises at least one alcohol; c) the active metal composition comprises cobalt and manganese; and d) the precipitating agent contains a carbonate, bicarbonate, phosphate, or borate.

### EXAMPLES

The following examples are put forth so as to provide those of ordinary skill in the art with a complete disclosure and description of how the compositions, catalysts, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

The following lab grade chemicals were used herein without further purification: Cobalt and Manganese salts (nitrates), sodium carbonate, ethanol, butanol, butane diol, and ethylene glycol.

### 1. Example 1—Catalyst A, Control Catalyst

0.6 g of silica was dissolved in 50 ml of demineralized water and stirred for an hour. This mixture was then heated to 80° C. 50 ml each of Co and Mn solutions (1M prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate (catalyst precursor) was first filtered and then washed several times with warm distilled water. The precipitate was then dried at 120° C. for 16 hours, and was subsequently calcined in static air in the furnace (500° C., 16 hours) to give the final catalyst. The specific formula for this material is CoMnBut_{0.0}/SiO₂. This catalyst is denoted by symbol “A.”

### 2. Example 2—Catalyst B

0.6 g of silica was dissolved in 40 ml of demineralized water and 10 ml of butanol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate (catalyst precursor) was first filtered and then washed several times with warm distilled water. The precipitate were then dried at 120° C. for 16 hours, and was subsequently calcined in static air in the furnace (500° C., 16 hours) to give the final catalyst. The specific formula for this material is CoMnBut₁₀/SiO₂. This catalyst is denoted by symbol “B.”

### 3. Example 3—Catalyst C

0.6 g of silica was dissolved in 25 ml of demineralized water and 25 ml of butanol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was then dried at 120° C. for 16 hours, and was subsequently calcined in static air in the furnace (500° C., 16 hours) to give the final catalyst. The specific formula for this material is COMnBut₂₅/SiO₂. This catalyst is denoted by symbol “C.”

### 4. Example 4—Catalyst D

0.6 g of silica was dissolved in 10 ml of demineralized water and 40 ml of butanol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was then dried at 120° C. for 16 hours, and was subsequently calcined in static air in the furnace (500° C., 16 hours) to give the final catalyst. The specific formula for this material is CoMnBut₄₀/SiO₂. This catalyst is denoted by symbol “D.”

### 5. Example 5—Catalyst E

0.6 g of silica was dissolved in 40 ml of demineralized water and 10 ml of butanol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. 20 ml of ethanol was added and stirred for an hour. The precipitate was then dried at 120° C. for 16 hours, and was subsequently calcined in static air in the furnace (500° C., 16 hours) to give the final catalyst. The specific formula for this material is CoMnBut_{10Et}/SiO₂. This catalyst is denoted by symbol “E.”

### 6. Example 6—Catalyst F

0.6 g of silica was dissolved in 40 ml of butanol and 10 ml of ethylene glycol. This mixture was stirred for an hour then heated up to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was then dried at 120° C. for 16 hours, and was subsequently calcined in static air in the furnace (500° C., 16 hours) to give the final catalyst. The specific formula for this material is CoMnEG₁₀/SiO₂. This catalyst is denoted by symbol “F.”

### 7. Example 7—Catalyst G

0.6 g of silica was dissolved in 20 ml of demineralized water and 80 ml of butanol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was then dried at 120° C. for 16 hours, and was subsequently calcined in static air in the furnace (500° C., 16 hours) to give the final catalyst. The specific formula for this material is CoMnBut₈₀/SiO₂. This catalyst is denoted by symbol “G.”

### 8. Example 8—Catalyst H

0.6 g of silica was dissolved in 20 ml of demineralized water and 80 ml of butanediol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was then dried at 120° C. for 16 hours, and was subsequently calcined in static air in the furnace (500° C., 16 hours) to give the final catalyst. The specific formula for this material is CoMnBD₈₀/SiO₂. This catalyst is denoted by symbol “H.”

### 9. Example 9—Catalyst I

0.6 g of silica was dissolved in 20 ml of demineralized water and 10 ml of ethylene glycol. This mixture was stirred for an hour then heated up to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was then dried at 120° C. for 16 hours, and was subsequently calcined in static air in the furnace (500° C., 16 hours) to give the final catalyst. The specific formula for this material is CoMnEG₈₀/SiO₂. This catalyst is denoted by symbol “I.”

### 10. Example 10—Catalyst J

0.6 g of silica was dissolved in 20 ml of demineralized water and 80 ml of ethanol. This mixture was stirred for an hour then heated to 80° C. 50 ml each of Co and Mn 1M solutions (prepared from the nitrate salts) were premixed and heated to 80° C. 1M of a sodium carbonate solution was prepared and heated to 80° C. Both solutions were added drop wise to the silica solution, which was continuously stirred whilst the temperature was maintained at 80° C. The pH was varied from 7.0 to 8.0. The precipitate was first filtered and then washed several times with warm distilled water. The precipitate was dried at 120° C. for 16 hours, and was subsequently calcined in static air in the furnace (500° C., 16 hours) to give the final catalyst. The specific formula for this material is CoMnEt₈₀/SiO₂. This catalyst is denoted by symbol “J.”

### 11. Results

The catalysts A-J described above were evaluated for their activity and selectivity along with short term as well as long studies of the catalyst stabilities. Prior to activity measurement, all of the catalysts were subjected to activation procedure which was performed at 350° C. with the ramp rate of 3° C. min⁻¹ for 16 hours in 50:50 H₂/N₂ flow (WHSV: 3600 h⁻¹). The catalytic evaluation was carried out in a high throughput fixed bed flow reactor setup housed in a temperature controlled system fitted with regulators to maintain pressure during the reaction. The catalysts performing experiments were performed after activation under the following conditions: 240° C., 5 bar, WHSV: 2500 h⁻¹, H₂/CO: 2, TOS 150 & 370 hours. The products of the reactions were analyzed through online gas chromatography analysis.

The performances of the catalysts are shown in Table 1.

TABLE 1

| | | | | | | | | | | |
|---|---|---|---|---|---|---|---|---|---|---|
| Catalyst | A | B | C | D | E | F | G | H | I | J |

CO Conversion (mol %)

| | | | | | | | | | |
|---|---|---|---|---|---|---|---|---|---|
| 65 | 70 | 68 | 75 | 69 | 64 | 70 | 76 | 68 | 73 |

Selectivity (mol %)

| | | | | | | | | | | |
|---|---|---|---|---|---|---|---|---|---|---|
| Olefins | 33 | 30 | 31.3 | 30 | 30.4 | 35 | 32 | 29 | 32 | 32 |
| Paraffins | 18 | 12 | 17 | 19 | 14 | 19 | 18 | 17 | 15 | 20 |
| CO₂ | 25 | 34 | 24 | 26 | 33 | 23 | 26 | 30 | 27 | 24 |
| Methane | 6 | 5 | 6 | 6 | 6 | 6 | 6 | 6 | 6 | 6 |
| Propylene | 9 | 10.4 | 10 | 9 | 11 | 11 | 10 | 10 | 11 | 10 |
| Alcohol | 9 | 10 | 8 | 8 | 11 | 8 | 10 | 10 | 10 | 9 |
| (T) | | | | | | | | | | |
| Olefin | 22 | 21 | 21.3 | 22.4 | 21 | 22.1 | 22 | 22.1 | 21.7 | 23.3 |
| Yield | | | | | | | | | | |

A described above, the base catalyst (A) containing Co and Mn was prepared following a conventional methodology using a conventional solvent (i.e. only water). The conversion of this catalyst (A) is around 65% with the total olefins selectivity at 33%. Catalysts B-E and G-J were prepared by a method disclosed herein to determine the effects of the use of a solvent comprising water and a non-water polar solvent.

Catalyst B was prepared with 40 ml water and 10 ml alcohol. The results show that the addition of alcohol increased the activity of the catalysts as the conversion is high, as well as the production of carbon dioxide, in catalyst B. This indicates that the catalysts have a deep activity making the complete oxidation of some of the products to the end product carbon dioxide. The preparation parameters were further altered to decrease the production of carbon dioxide without compromising the activity of catalysts. This was achieved by altering the ratio of alcohol to water, to use 40 ml of alcohol and 10 ml of water (catalyst D), and the activity increased about 10% as compared to the base catalyst, and a decrease in the carbon dioxide to 10%, which is similar to the base catalyst. Further the effect of other alcohols like ethanol, ethylene glycol and butane diol during catalyst preparation were also evaluated, see Table 1. FIG. 1 and FIG. 2 show the complete carbon distribution of olefins of catalysts A-J.

What is claimed is:

1. A method comprising the step of:
a) mixing a solution comprising a catalyst support and a solvent comprising water and a non-water polar solvent with an active metal composition and a precipitating agent, thereby producing a catalyst precursor.

2. The method of claim 1, wherein the method further comprises the steps of:
b) drying the catalyst precursor; and
c) calcining the catalyst precursor, thereby producing a catalyst.

3. The method of claim 1, wherein the solution has a temperature from about 20° C. to about 95° C.

4. The method of claim 1, wherein the solution has a temperature from about 60° C. to about 90° C.

5. The method of claim 1, wherein the catalyst support comprises Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof.

6. The method of claim 5, wherein the catalyst support comprises SiO₂.

7. The method of claim 1, wherein the non-water polar solvent comprises at least one alcohol.

8. The method of claim 7, wherein the at least one alcohol comprises methanol, ethanol, butanol, butane diol, pentanol, hexanol ethylene glycol, propylene glycol, dipropylene glycol, tripropylene glycol, or butylene glycol, or a mixture thereof.

9. The method of claim 7, wherein the at least one alcohol comprises butanol.

10. The method of claim 1, wherein the volume ratio of the water:non-water polar solvent in the solvent is from about 1:10 to about 1.1.

11. The method of claim 1, wherein the concentration of the catalyst support in the solution is at least 0.0001 g catalyst support per ml of solvent.

12. The method of claim 1, wherein the active metal composition comprises a cobalt and manganese.

13. The method of claim 1, wherein the precipitating agent comprises a carbonate, bicarbonate, phosphate, or borate.

14. The method of claim 1, wherein the solution has a basic pH.

15. The method of claim 1, wherein the catalyst has the formula CoMnₓS_{y}O_{z}, wherein S is a catalyst support, wherein the molar ratio of x is from about 0.8 to about 1.2; wherein the molar ratio of y is from about 0.01 to about 5.0; and wherein the molar ratio of z is a number determined by the valence requirements of Co, Mn, and S.

16. A catalyst precursor produced by the method of claim 1.

17. A catalyst produced by the method of claim 2.

18. A method of producing C2+ hydrocarbons comprising contacting syngas with the catalyst of claim 2, thereby producing C2+ hydrocarbons.

19. A composition comprising:
a) a catalyst support;
b) a solvent comprising water and a non-water polar solvent;
c) an active metal composition; and
d) a precipitating agent.

20. The composition of claim 19, wherein
a) the catalyst support comprises Al₂O₃, SiO₂, TiO₂, CeO₂, AlPO₄, ZrO₂, MgO, ThO₂, boehmite, silicon-carbide, Molybdenum-carbide, an alumino-silicate, kaolin, a zeolite, or a molecular sieve, or a mixture thereof;
b) the non-water polar solvent comprises at least one alcohol;
c) the active metal composition comprises cobalt and manganese; and
d) the precipitating agent contains a carbonate, bicarbonate, phosphate, or borate.
